(19) United States
(12) Patent Application Publication
(10) Pub. No.: US 2019/0091075 A1
(43) Pub. Date: Mar. 28, 2019

(54) Male Incontinence Device

(71) Applicant: Clarence H. JONES, JR., Humble, TX (US)

(72) Inventor: Clarence H. JONES, JR., Humble, TX (US)

(21) Appl. No.: 15/718,699

(22) Filed: Sep. 28, 2017

(51) Int. Cl. A61F 13/471; A61F 13/58

(52) U.S. Cl. CPC A61F 13/471; A61F 2013/15121; A61F 13/58

(57) ABSTRACT

This male incontinence device provides a water-proof sleeve into which the penis is inserted. This sleeve contains an inner permeable membrane covering an absorbent micro-fiber material to collect excreted urine. The sleeve is long enough to collect between 7 and 16 fl. oz. of urine before needing to be removed and discarded. Because of its size and shape, the profile of the sleeve while being worn appears natural.

### CROSS-REFERENCE TO RELATED APPLICATIONS

This application claims priority to U.S. Provisional Application No. 62/400,949, filed Sep. 28, 2016.

No federally sponsored research applies to this application.

There are no related research partners for this application

There are no Sequence or Computer Codes filed with this application.

### BACKGROUND OF THE INVENTION

The present device relates to a male incontinence medical assistance device; more specifically to a device to capture urine from men who suffer from male incontinence either as a result of injury, surgery, or aging.

### BRIEF SUMMARY OF THE INVENTION

The present invention relates to a male incontinence device comprising a sleeve providing a single opening having an impermeable exterior layer, an absorbent micro-fiber middle layer, and a permeable inner layer; and, an adhesive strip extending along the exterior surface of the impermeable sleeve providing a means for resisting movement of the sleeve to a distal end of the sleeve. The sleeve of this device contains the absorbent micro-fiber middle layer of the impermeable sleeve fabricated from polyesters, polyamides, or a conjugation of polyester, polyamide, and polypropylene.

Alternatively, the male incontinence device can contain the absorbent organic material to collect the urine.

The sleeve can be formed from two layered sheets of a water-proof polymer, such as a polyethelene, each providing an impermeable exterior layer, a micro-fiber middle layer, and a permeable inner layer, each layered sheet joined together with each permeable inner layer adjacent the other permeable inner layer, and sealed together with a heat crimp, sewing, or gluing along the edge of each sheet to form the sleeve.

The male incontinence device provides a sleeve that holds between about 7 fl. oz. to about 16 fl. oz. of urine.

### BRIEF DESCRIPTION OF THE SEVERAL VIEWS OF THE DRAWING

FIG. 1 is a side perspective view of the male incontinence device showing the opening at a proximal end of the sleeve and providing a sealed exterior edges for containing urine.

FIG. 2 is a frontal view of the male incontinence device showing the proximal end of the sleeve and the sealed distal end of the sleeve.

FIG. 3 is a side view of the male incontinence device showing the opening at the proximal end of the sleeve and a side view of the sealed sleeve.

FIG. 4 is a posterior view of the male incontinence device showing the opening at the proximal end of the sleeve and the adhesive tape which may be selectively removed to permit the adhesive material on the exterior surface to support the sleeve in underpants or garments of the wearer of the device.

FIG. 5 is a top perspective view of the male incontinence device showing the opening at the proximal end of the sleeve and the folded sleeve prior to full engagement of the sleeve in a vertical position.

FIG. 6 shows a frontal perspective view of the male incontinence device showing the proximal edge at the of the device prior to full engagement of the sleeve in a vertical position.

FIG. 7 is a perspective view of the male incontinence device engaging the underpants of the incontinent male.

FIG. 8 is a sectional view of the layers of the male incontinence device showing the impermeable outer layer, the absorbent micro-fiber inner layer, covered by the permeable inner layer permitting the urine to be collected by the absorbent inner layer while keeping the penis of the wearer dry.

### DETAILED DESCRIPTION OF THE INVENTION

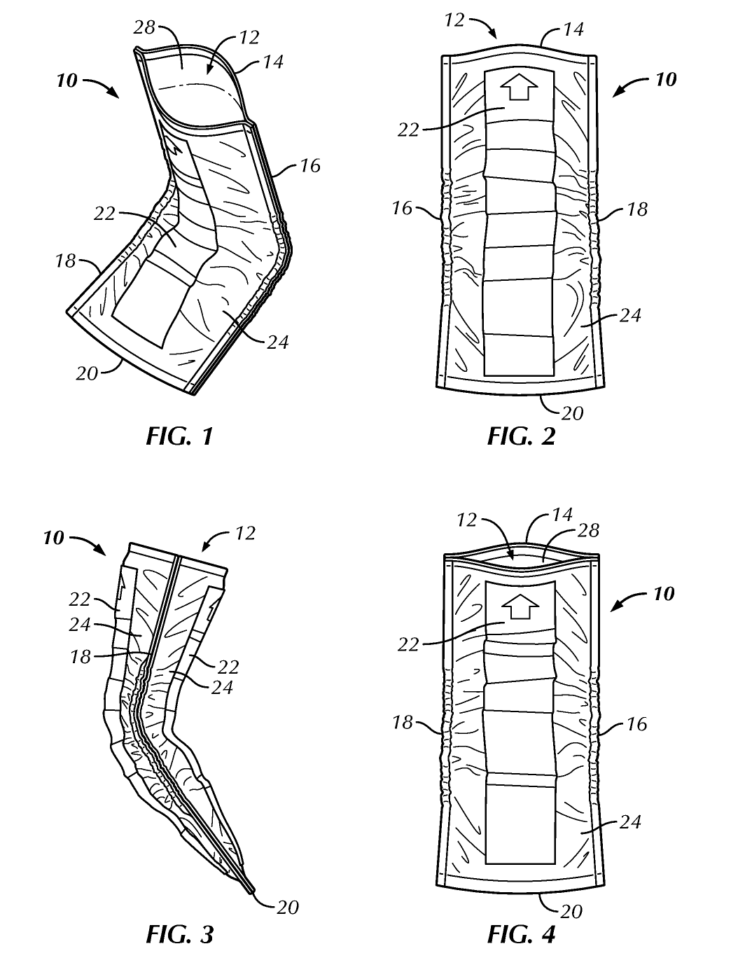

FIG. 1 shows a perspective view of the male incontinence sleeve 10 of the present invention providing an open end 12 sufficient to allow a penis to be inserted. The layered material 28 provides sealed edge 14. The lateral edges 16, 18, together with the bottom edge 20 form the closed sealed sleeve 10. The outer material 24 of the sleeve 10 is fabricated with an impermeable membrane material providing a seal against the leaking of urine from the interior of the sleeve 10. A segmented, tabbed strip 22 is placed over an exterior adhesive material (not shown) on both sides of the sleeve 10 to enable the user to remove part or all of the tabbed strip 22 allowing the underlying adhesive to contact the underwear of the user. The lateral edges 16, 18, provide a bias allowing the sleeve to fold between the legs of the wear and conform to the interior surface of the underwear of the user. Alternatively, the sleeve 10 can be fabricated as a unitary sleeve (not shown) without the need to connect two sheets shown in the drawings of FIGS. 1-6. In this embodiment, the multi-layered material would surround the penis of the user.

FIG. 2 is a frontal view of the sleeve 10. Each of the elements described in FIG. 1 are shown in FIG. 2. Similarly, FIG. 3 shows a side view of the sleeve 10 showing the segmented, tabbed strip 22 on both sides of the sleeve 10. FIG. 4 is a posterior view of the sleeve 10. Each of the elements described in FIG. 1 are shown on this posterior view of the sleeve 10.

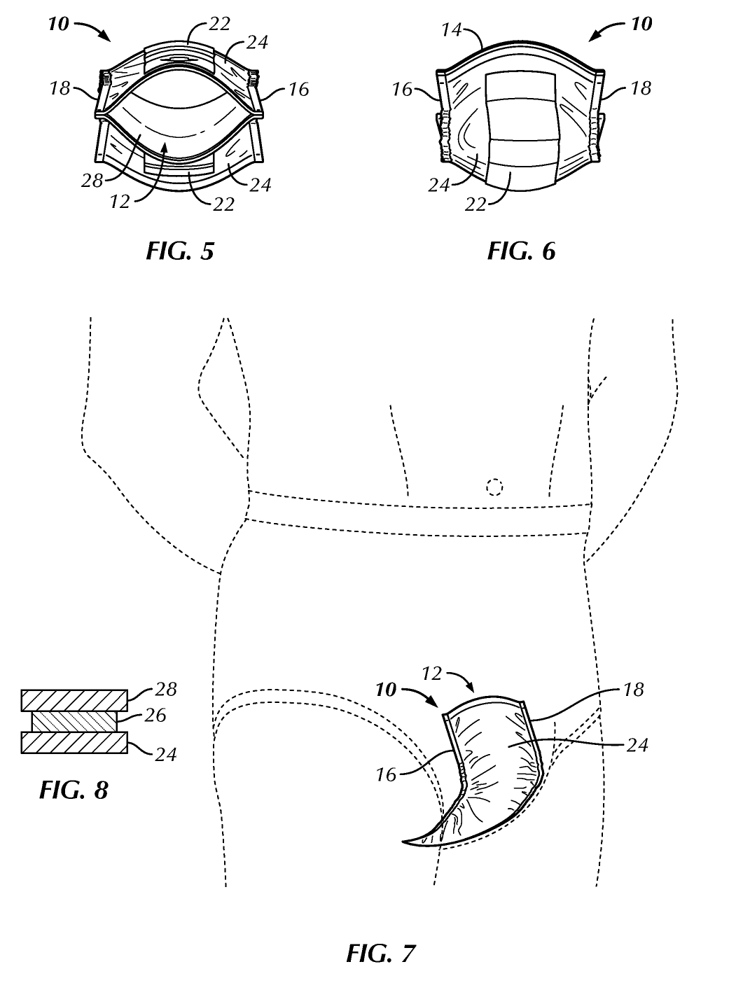

FIG. 5 is a top perspective view of the sleeve 10 showing the open end 12 of the sleeve 10 in its folded, biased position. FIG. 6 is a similar bottom view of the sleeve 10 showing the folded sleeve, which permits the sleeve 10 to be worn between the legs of the user. FIG. 7 shows the sleeve 10 inserted on the inside of the user's underwear, extending between the legs of the user, with the tabbed strip 22 removed allowing the adhesive (not shown) on the exterior surface 24 of the sleeve 10 to support the device in the underwear of the user. Biasing means can be attached to the edge of the sleeve 10 to cause the device to bend under the user's legs.

FIG. 8 is a cross-sectional view of the multi-layered material used to fabricate the male incontinence device of the present invention. The outer or exterior layer 24 carries the absorbent micro-fiber central layer 26. The inner membrane 28 allows urine to pass into the absorbent central layer 26 where it remains and is retained. The absorbent central layer can be fabricated with sufficient space to allow at least about 7 fl. oz. to about 16 fl. oz. of urine to be absorbed. Once filled, the user can then remove the sleeve 10 without removal of his lower clothing and dispose of the used sleeve in any trashcan.

Alternatively, the user can use a reusable strap (not shown) that encircles his waist while attached to the adhesive portion 22 of the sleeve 10 to permit use of device without the need of underwear.

1. A male incontinence device comprising:
a sleeve providing a single opening having an impermeable exterior layer, an absorbent micro-fiber middle layer, and a permeable inner layer; and,
an adhesive strip extending along the exterior surface of the impermeable sleeve providing a means for resisting movement of the sleeve to a distal end of the sleeve.

2. The male incontinence device of claim 1 wherein the absorbent micro-fiber middle layer of the impermeable sleeve is fabricated from polyesters, polyamides, or a conjugation of polyester, polyamide, and polypropylene.

3. The male incontinence device of claim 1 wherein the absorbent interior layer is an absorbent organic material.

4. The male incontinence device of claim 1 wherein the sleeve is formed from two layered sheets of polyethelene providing an impermeable exterior layer, a micro-fiber middle layer, and a permeable inner layer, each layered sheet joined together with each permeable inner layer adjacent the other permeable inner layer, and sealed together with a heat crimp along the edge of each sheet to form the sleeve.

5. The male incontinence device of claim 1 wherein the sleeve is formed from two layered sheets of polyethelene providing an impermeable exterior layer, a micro-fiber middle layer, and a permeable inner layer, each layered sheet joined together with each permeable inner layer adjacent the other permeable inner layer, and sealed together by sewing the edge of each sheet to form the sleeve.

6. The male incontinence device of claim 1 wherein the sleeve is formed from two layered sheets of polyethelene providing an impermeable exterior layer, a micro-fiber middle layer, and a permeable inner layer, each layered sheet joined together with each permeable inner layer adjacent the other permeable inner layer, and sealed together by gluing the edge of each sheet to form the sleeve.

7. The male incontinence device of claim 1 wherein the sleeve holds between about 7 fl. oz. to about 16 fl. oz. of urine.
